(19) United States
(12) Patent Application Publication
(10) Pub. No.: US 2024/0206881 A1
(43) Pub. Date: Jun. 27, 2024

(54) NEUROVASCULAR OCCLUSION DEVICE

(71) Applicant: Embolic Acceleration, LLC, Miramar, FL (US)

(72) Inventors: Andrew H. Cragg, Edina, MN (US); John Logan, Plymouth, MN (US); Brett E. Naglreiter, Hollywood, FL (US); Alejandro Espinosa, Miami, FL (US)

(21) Appl. No.: 18/364,410

(22) Filed: Aug. 2, 2023

Related U.S. Application Data
(63) Continuation of application No. 16/454,943, filed on Jun. 27, 2019, now abandoned.
Application No. 16/454,943 is a continuation of application No. 15/273,521, filed on Sep. 22, 2016, now abandoned.
(60) Provisional application No. 62/232,321, filed on Sep. 24, 2015.

(51) Int. Cl. A61B 17/12; A61B 17/00; A61B 90/00

(52) U.S. Cl. CPC A61B 17/12145; A61B 17/12022; A61B 17/12031; A61B 17/12109; A61B 17/12172; A61B 17/12177; A61B 2017/00867; A61B 2017/12054; A61B 90/39

(57) ABSTRACT

A neurovascular occlusion device for occluding a vessel and a delivery system for delivering the occlusion device. The occlusion device can include a self-expandable support structure defining a concave occlusive component and an anchoring component separated by a neck component. The occlusive component can carry a functionally occlusive membrane configured to prevent substantially all fluid from flowing past the occlusion device when the occlusion device is expanded in the vessel.

### RELATED APPLICATIONS

This application is a continuation of U.S. patent application Ser. No. 16/454,943, filed Jun. 27, 2019, which is a continuation of U.S. patent application Ser. No. 15/273,521, filed Sep. 22, 2016, which claims priority benefit of U.S. Patent Application No. 62/232,321, filed Sep. 24, 2015, which is hereby incorporated by reference in its entirety herein. This application is also related to at least U.S. Publication No. 2015/0039017, filed Jul. 31, 2014, titled “METHODS AND DEVICES FOR ENDOVASCULAR EMBOLIZATION,” which is hereby incorporated by reference in its entirety herein.

### BACKGROUND

### Field

The present disclosure generally relates to apparatuses and methods for occluding blood flow.

### Description of the Related Art

Various vascular devices have been proposed to occlude blood flow for various applications in the vascular system. Early devices used inflatable balloons in order to block vessels. However, the use of balloons in the intracranial vasculature presents specific challenges. For example, balloons generally exhibit low trackability, meaning that they are difficult to navigate, especially through tortuous vessels, such as those commonly found in the intracranial circulation. In addition, premature (i.e., non-intentional) detachment from the delivery device can lead to adverse consequences such as cerebral artery blockage and stroke. Even once in place, balloons can move forward during the process of inflation, making placement of the unexpanded balloon in order to achieve precise positioning after inflation relatively difficult. Balloons that dislodge and migrate can require open skull surgery especially where the balloon has become lodged in a major vessel, for example, in a cerebral artery.

An alternative approach has been to use hydrogel-coated coils in order to produce rapid vascular occlusion. However, there remains a significant period between placement of the coil and formation of the occlusive clot, even when using coated coils. This leads to concern that during formation of the clot, distal clot migration can occur, with potentially devastating consequences such as stroke. Further, the geometric configuration and unpredictability of coil-based embolization prevents precise occlusion of a short vascular segment.

Thus, notwithstanding the various efforts in the past, there remains a need for devices and methods for rapid, well-controlled, safe, and effective vessel occlusion.

### SUMMARY

Certain aspects of the disclosure are directed toward a neurovascular occlusion device for occluding blood flow in a vessel. The occlusion device can include a support structure that self-expands from a reduced cross-section for transluminal navigation to an enlarged cross-section for occluding a vessel. The support structure can have an expansion ratio of at least about 8:1. The support structure can define a concave occlusive component and an anchoring component separated by a neck component. The support structure can be symmetric about a plane extending through a longitudinal midpoint of the occlusion device when the support structure is expanded to the enlarged cross-section.

Certain aspects of the disclosure are directed toward a neurovascular occlusion device for occluding blood flow in a vessel. The occlusion device can include a support structure that self-expands from a reduced cross-section for transluminal navigation to an enlarged cross-section for occluding a vessel. The support structure can define a concave occlusive component and an anchoring component separated by a neck component. The occlusive component can carry a functionally occlusive membrane configured to prevent substantially all fluid from flowing past the occlusion device when the occlusion device is expanded to the enlarged cross-section in the vessel, e.g., preventing at least about 90% or 95% of fluid flowing past the occlusion device at 120 mmHg pressure.

Certain aspects of the disclosure are directed toward a neurovascular occlusion device for occluding blood flow in a vessel. The occlusion device can include a support structure that self-expands from a reduced cross-section for transluminal navigation to an enlarged cross-section for occluding a vessel. The support structure can define a concave occlusive component and an anchoring component separated by a neck component. A first ratio of wall space to open area in the neck component can be smaller than a second ratio of wall space to open area in the occlusive component or the anchoring component to permit the occlusion device to conform to an arcuate portion of the vessel.

Certain aspects of the disclosure are directed toward methods of occluding a vessel using any of the occlusion devices described herein. The method can include advancing a pusher assembly into the vessel. The pusher assembly can include an outer diameter of less than or equal to about 3F. The pusher assembly can include an interlock pusher having a distal region, an intermediate region, and a proximal region. The method can further include releasing the occlusion device from the pusher assembly. The occlusion device can include a first interference surface configured to interface with a second interference surface of the distal region of the pusher assembly until the occlusion device is released from the pusher assembly.

Any feature, structure, or step disclosed herein can be replaced with or combined with any other feature, structure, or step disclosed herein, or omitted. Further, for purposes of summarizing the disclosure, certain aspects, advantages, and features of the inventions have been described herein. It is to be understood that not necessarily any or all such advantages are achieved in accordance with any particular embodiment of the inventions disclosed herein. No individual aspects of this disclosure are essential or indispensable.

### BRIEF DESCRIPTION OF THE DRAWINGS

Various embodiments are depicted in the accompanying drawings for illustrative purposes, and should in no way be interpreted as limiting the scope of the embodiments. Furthermore, various features of different disclosed embodiments can be combined to form additional embodiments, which are part of this disclosure.

FIG. 1A illustrates an embodiment of an occlusion device.

FIG. 1B illustrates a proximal end view of the embodiment of the occlusion device shown in FIG. 1A.

FIG. 1C illustrates a distal end view of the embodiment of the occlusion device shown in FIG. 1A.

FIG. 2A illustrates a side view of a support structure of the occlusion device shown in FIG. 1A.

FIG. 2B illustrates an alternative side view of the support structure shown in FIG. 2A.

FIG. 3 illustrates another embodiment of a support structure that can form a portion of the occlusion device.

FIG. 4 illustrates a side view of another embodiment of a support structure.

FIG. 5 illustrates a portion of a pusher assembly configured to deploy the occlusion device shown in FIG. 1A.

FIG. 6A illustrates a distal portion of an interlock pusher interfacing with a proximal portion of the occlusion device.

FIG. 6B illustrates a distal portion of the interlock pusher shown in FIG. 6A without the occlusion device.

FIG. 6C illustrates a cross-section of the interlock pusher shown in FIG. 6B.

### DETAILED DESCRIPTION

### Occlusion Device

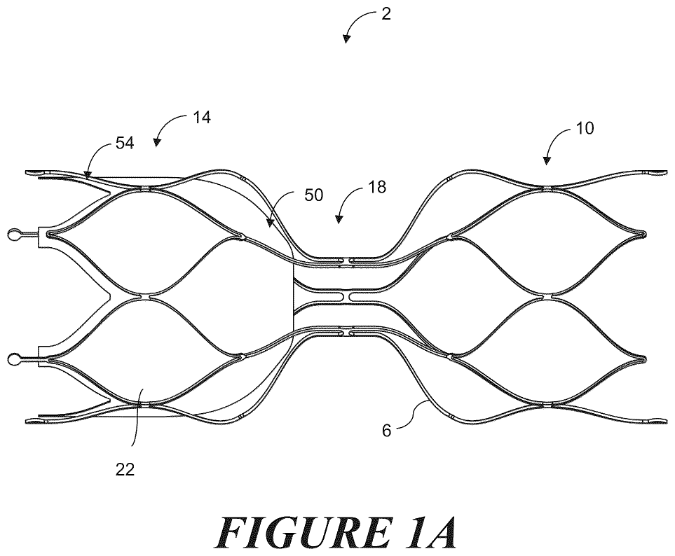

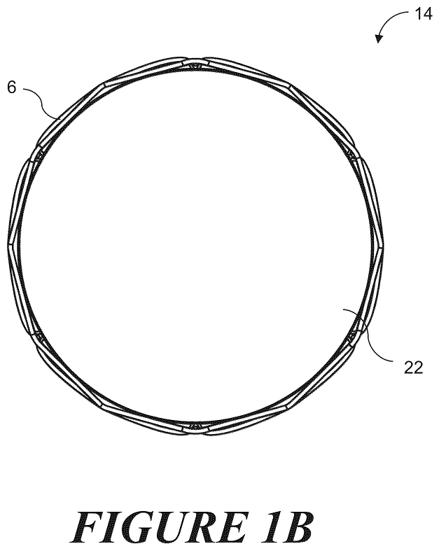

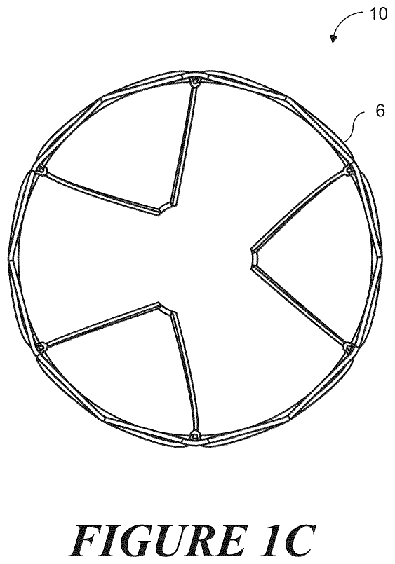

FIGS. 1A-1C illustrate an occlusion device 2 for occluding blood flow in a vessel. The occlusion device 2 can include a self-expanding support structure 6 including an anchoring portion 10 and an occlusive portion 14 separated by a neck portion 18. The occlusive portion 14 can be concave in a first direction and the anchoring portion 10 can be concave in an opposite direction to form a generally hourglass shape. When the occlusion device 2 is deployed in the vessel, the occlusive portion 14 can be positioned proximally and be concave in an upstream flow direction, and the anchoring portion 10 can be positioned distally and be concave in the downstream flow direction. The occlusion device 2 can be configured such that blood pressure against the occlusive portion 14 provides a radially outwardly directed force to seal the occlusive portion 14 against the vessel wall and an axially directed force against the neck portion 18 which increases a radial force between the anchoring portion 10 and the vessel wall. Accordingly, increased blood pressure enhances the seal of the occlusive portion 14 and increases the radial outward force anchoring the anchoring portion 10 against the vessel wall.

The occlusive portion 14 can be covered with an occlusive membrane 22 to occlude substantially all fluid from flowing past the occlusion device 2. For example, the occlusion device 2 can achieve a reduction in blood flow of at least about 80% within about five minutes of deployment in a blood vessel, preferably within about two minutes of deployment from the tube in a blood vessel or within about one minute of deployment in a blood vessel, without reliance on biological processes to achieve occlusion (e.g., based on the mechanical occlusion of the occlusion device 2). In certain aspects, the occlusion device 2 can be configured to achieve total occlusion within about five minutes of deployment in a blood vessel or within about two minutes of deployment in a blood vessel, preferably within about one minute of deployment from the tube in a blood vessel. The rate of occlusion can be measured according to the Occlusion Protocol described in U.S. Publication No. 2015/0039017, filed Jul. 31, 2014, titled “METHODS AND DEVICES FOR ENDOVASCULAR EMBOLIZATION,” which is included in the Appendix.

The anchoring portion 10 can remain uncovered to provide stability and prevent migration. For example, the occlusion device 2 can exhibit a migration of less than about 5.0 mm for at least 10 minutes under pressures of at least about 55 mmHg and/or less than or equal to about 300 mmHg, for example, between about 100 mmHg and 150 mmHg, between about 150 mmHg and about 300 mmHg, between about 200 mmHg and about 300 mmHg, between about 250 mmHg and about 300 mmHg, such as about 270 mmHg, as determined by the Migration Protocol described in U.S. Publication No. 2015/0039017, filed Jul. 31, 2014, titled “METHODS AND DEVICES FOR ENDOVASCULAR EMBOLIZATION,” which is included in the Appendix.

Clinically, it can be desirable for the occlusion device 2 to exert sufficient radial outward pressure to maintain proper vessel wall apposition and resist migration of the occlusion device 2 after deployment. The occlusion device 2 can exert a radial outward pressure between about 30 mmHg and about 50 mmHg, for example, between about 30 mmHg and about 40 mmHg, between about 35 mmHg and about 45 mmHg, or between about 40 mmHg and about 50 mmHg at the diameter of an intended target site in a vessel.

Blood pressure acting on the occlusive portion 14 encourages the occlusive portion 14 of the support structure 6 to open and exert more radial outward force on the blood vessel wall and transmits force axially through the support structure 6 to the distal anchoring portion 10, which also increases radial outward force on the distal anchoring portion 10. The radial outward force at the occlusive and anchoring portions 14, 10 of the occlusion device 2 can increase by up to 20 mmHg, for example, between about 10 mmHg to about 15 mmHg, or between about 15 mmHg and about 20 mmHg.

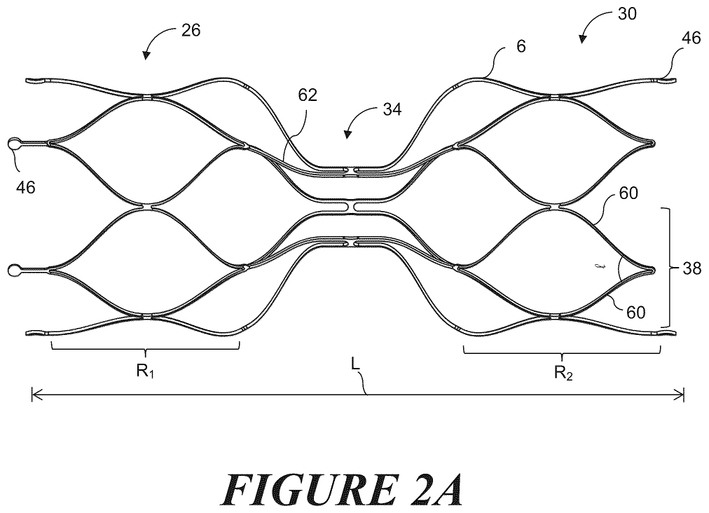

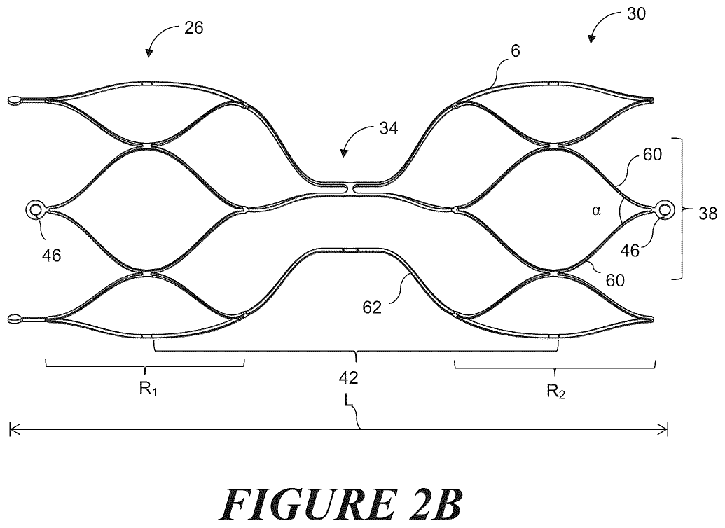

FIGS. 2A and 2B illustrate the support structure 6 in an unconstrained configuration. The support structure 6 can be symmetric about a transverse plane extending through a mid-point along a length L of the occlusion device 2 in the constrained and/or unconstrained configurations, e.g., a length of an occlusive component 26 of the support structure 6 can be the same as a length of an anchoring component 30 of the support structure 6, and/or a shape of the occlusive component 26 of the support structure 6 can be the same as a shape of the anchoring component 30 of the support structure 6.

The support structure 6 can include any of a number of medical grade materials, including, but not limited to, polymers (e.g., PET) or non-ferrous metals (e.g., nitinol, stainless steel, or cobalt chrome). For example, in the illustrated embodiment, the support structure 6 can be laser-cut from nitinol hypotube to form the strut configuration shown in FIGS. 2A and 2B. Each strut 60 can have a diameter between about 0.02 mm and about 0.13 mm, e.g., between about 0.025 mm and about 0.05 mm.

Each of the occlusive component 26 and the anchoring component 30 can include a single ring R₁, R₂ of cells 38 (e.g., diamond-shaped cells). The struts 60 forming each cell 38 can extend at an angle relative to the direction of blood flow (e.g., at an angle between about 30 degrees and about 60 degrees relative to the direction of blood flow or at an angle between about 45 degrees and about 75 degrees relative to the direction of blood flow). The angle α formed between adjacent struts 60 of a cell 38 can form an angle between about 70 degrees and about 130 degrees, for example, between about 70 degrees and 90 degrees, between about 80 degrees and about 100 degrees, between about 90 degrees and about 110 degrees, between about 100 degrees and about 120 degrees, or between about 110 degrees and about 130 degrees. The unconstrained pore size of each cell 38 can be less than or equal to about: 1.5 mm, 1.25 mm, 1.0 mm, or otherwise.

As shown in FIGS. 2A and 2B, each cell 38 of the occlusive component 26 can be longitudinally aligned with a cell 38 of the anchoring component 30. However, in other configurations the ring R₁ of cells 38 of the occlusive component 26 can be circumferentially offset from the ring R₂ of cells 38 of the anchoring component 30, such that none of the cells are longitudinally aligned. Further, although not shown, additional rings of cells (e.g., two, three, or more) can be added to the occlusive component 26 and/or the anchoring component 30 of the support structure 6.

The ring R₁ of cells 38 of the occlusive component 26 can be connected to the ring R₂ of cells 38 of the anchoring component 30 by a series of struts 62 extending through a neck component 34 of the support structure 6. The struts 62 can be configured to facilitate expansion of the neck component 34 and form cells 42 in the neck component 34.

In some configurations, a cell 42 in the neck component 34 can have greater open area than a cell 38 in the occlusive component 26 or the anchoring component 30, e.g., at least about: 1.5× greater, 2× greater, or more. Since the neck component 34 has a small ratio of wall space (e.g., strut area) to open area, the neck component 34 can impart sufficient flexibility to permit the occlusion device 2 to conform to an arcuate portion of the vessel, while avoiding the creation of a focal point for pressure, which can be detrimental in the neurovasculature. The ratio of wall space to open area in the neck component 34 can be less than the ratio of wall space to open area in the occlusive component 26 or the anchoring component 30. For example, the ratio of wall space to open area in the neck component 34 can be less than or equal to about: 10%, 8%, 6%, 4%, or otherwise. The ratio of wall space to open area in the occlusive component 26 or the anchoring component 30 can be less than or equal to about: 15%, 12%, 10%, 8%, or otherwise. In some scenarios, the ratio of wall space to open area in the neck component 34 can be about between about 4% and 6%, while the ratio of wall space to open area in the occlusive component 26 or the anchoring component 30 can be between about 8% and 10%.

In other configurations, it is imaginable that a cell 38 in the occlusive component 26 or the anchoring component 30 can have a greater open area than a cell 42 in the neck component 34, e.g., at least about: 1.5× greater, 2× greater, or more, such that a ratio of wall space (e.g., strut area) to open area in the neck component 34 is greater than the ratio of wall space to open area in the occlusive component 26 or the anchoring component 30. The larger cells in the occlusive component 26 and/or the anchoring component 30 permit those regions of the occlusion device 2 to expand to a larger diameter than the neck component 34.

The support structure 6 can self-expand from a reduced cross-section for transluminal navigation to an enlarged cross-section for occluding a vessel. The expansion ratio of the support structure 6 can be configured to permit the occlusion device 2 to compress to a minimum size suitable for delivery through a microcatheter 104 having an inner diameter of less than or equal to about: 1.0 mm, 0.75 mm, 0.6 mm, or 0.5 mm, thereby minimizing trauma to the vessel during delivery. Further, the expansion ratio can be configured such that a single, expanded occlusion device 2 is capable of preventing substantially all fluid from flowing past the occlusion device 2 in vessel range of different sized target vessels, e.g., between about 1.5 mm and about 6.0 mm, such as between about 1.5 mm and about 2.5 mm, between about 3.0 mm and about 4.0 mm, between about 3.5 mm and about 4.5 mm, between about 4.0 mm and about 5.0 mm, between about 3.0 mm and about 6.0 mm, or other ranges in between. Although, additional occlusion devices 2 (e.g., two, three, or more) can be delivered depending on clinical judgment.

The support structure 6 can have an expansion ratio of at least about: 6:1, 7:1, 8:1, 9:1, 10:1, 11:1, or otherwise. For example, the support structure 6 can have an expansion ratio between about 6:1 and about 8:1, between about 7:1 and about 9:1, between about 8:1 and about 10:1, between about 9:1 and about 11:1, or otherwise.

The aspect ratio of the occlusion device 2 when comparing unconstrained diameter to unconstrained length can be less than or equal to about: 2:1, 1.5:1, 1:1, or otherwise, such as between about 1:1 and about 1.5:1. The aspect ratio of the occlusive portion 14 of the occlusion device 2 when comparing unconstrained diameter to unconstrained length can be at least about: 1.5:1, 2:1, 2.5:1, or otherwise, such as between about 1.5:1 and about 2.5:1. A shorter occlusive length may be helpful to preserve patency of a branch vessel that is just proximal to the target occlusion site to avoid occluding a side branch or other vessel that provides collateral blood flow.

The maximum constrained diameter of the occlusion device 2 can be less than or equal to about 1.0 mm, for example between about 0.5 mm and about 1.0 mm. The unconstrained diameter can be between at least about 1× a diameter of the vessel in which the occlusion device 2 is deployed, or at least about: 1.2×, 1.3×, 1.4×, or 1.5× the diameter of the vessel, such as between about 1.2× and about 1.5×. The unconstrained diameter of the occlusion device 2 can be less than or equal to about: 20.0 mm, 10.0 mm, 6.0 mm, 5.0 mm, 4.0 mm, or otherwise. For example, the unconstrained diameter can be between about 4.0 mm and about 6.0 mm.

The maximum constrained length of the occlusion device 2 can be less than or equal to about 30.0 mm. The unconstrained length compared to the diameter of the vessel in which the occlusion device 2 has been deployed can be less than or equal to about: 2×, 1.5×, 1×, 0.75×, 0.5×, 0.25×, or otherwise. For example, the unconstrained length of the occlusion device 2 can be between about 5.0 mm and about 20.0 mm, such as between about 5.0 mm and about 10.0 mm, between about 10.0 mm and about 15.0 mm, or between about 15.0 mm and about 20.0 mm.

One or more radiopaque markers 46 can be attached to the support structure 6 to facilitate visualization of the occlusion device 2. For example, one or more markers 46 (e.g., one, two, three, or more) can be positioned at the proximal and/or distal end of the support structure 6, so that the ends of the occlusion device 2 can be visualized. The markers 46 can be attached (e.g., press-fit, welded, glued, etc.) onto the strut endings at either end of the support structure 6. As shown in FIGS. 2A and 2B, the markers 46 can include a diameter that is greater than a diameter of the strut 60 to facilitate visualization. At least some of the markers 46 can include an aperture (eyelet, opening, or the like). Alternative marker configurations can be imagined so long as the marker is configured to interlock with a corresponding feature of the pusher assembly (described in further detail below). For example, the marker can be generally T-shaped having a first portion and a second portion. A major axis of the first portion can be generally perpendicular to a major axis of the second portion. The second portion can be positioned longitudinally outward from the first portion. As another example, the marker can be generally Z-shaped. The Z-shaped marker can have a first end portion, a second end portion generally parallel to the first end portion, and an intermediate portion extending from an end of the first end portion to a circumferentially offset end of the second end portion. The major axes of the first and second end portions can be generally parallel or perpendicular to a longitudinal axis of the occlusion device. As yet another example, the marker can be serrated. As another example, the marker configuration can be rectangular with the long axis aligned axially to maximize the amount of radiopaque material without increasing the device profile while compressed in the delivery system.

As described above, at least a portion of the support structure 6 can be covered with an occlusive membrane 22. The occlusive membrane 22 can form a concave shape (e.g., dome shape) that is functionally occlusive (see FIGS. 1A-1C). The functionally occlusive membrane can prevent more than 10% (or more than 20%, 30%, 40%, 50%, 60%, 70%, or 80%) of fluid from flowing past the occlusion device at 120 mmHg pressure within five minutes, within three minutes, within thirty seconds, or within five seconds. It may be desirable to provide a functionally occlusive membrane that can achieve mechanical occlusion of blood flow in a vessel without requiring biological processes. Thus, in some embodiments, the occlusive membrane 22 can prevent more than 90% of fluid from flowing past the occlusion device at 120 mmHg pressure within five minutes, within three minutes, within thirty seconds, or within five seconds of deploying the occlusion device 2 without reliance on biological processes. A functionally occlusive membrane 22 can be achieved by providing an occlusive membrane 22 without any openings (e.g., for a guidewire) that are greater than an average pore size of the occlusive membrane 22, which can be less than or equal to about: 0.2 mm, 0.15 mm, 0.125 mm, 0.1 mm, or otherwise. Since the occlusion device 2 does not have to be delivered over the wire (as explained further below), the occlusion device 2 does not require a thru-lumen.

The thickness of the occlusive membrane 22 can be generally the same along the length of the occlusive membrane 22. However, a dome area 50 of the occlusive portion 14 (e.g., at the region of curvature) and the region 54 near the open end of the occlusive portion 14 (e.g., at the cylindrical region) can have a different number of layers. The dome area 50 can have fewer layers than the region 54, e.g., the region 54 can have multiple layers (e.g., two, three, or more) that collectively have a same thickness as the dome area 50.

In other embodiments, the thickness of the occlusive membrane 22 can vary, e.g., between about 10 microns and 30 microns, along a length of occlusive portion 14. For example, a thickness of the occlusive membrane 22 can be thicker at a dome area 50 of the occlusive portion 14 (e.g. at the region of curvature) compared to region 54 near the open end of the occlusive portion 14 (e.g., at the cylindrical region).

The occlusive membrane 22 can have sufficient tensile strength to resist yielding, stretching, or breaking under at least normal blood pressures, e.g., at least about: 120 mmHg, 140 mmHg, 160 mmHg, or otherwise.

Possible materials for the occlusive membrane 22 can include, but are not limited to PTFE, PET, silicone, latex, TecoThane, nylon, PET, Carbothane (Bionate), fluoropolymers (e.g., PVDF), SIBS, TecoFlex, Pellethane, Kynar, or PGLA. The occlusive membrane 22 can be covered with a material to at least temporarily inhibit thrombus formation (e.g., a hydrophilic covering), so the occlusion device 2 can be retracted and repositioned prior to final placement.

The occlusive membrane 22 can be formed using an electrospinning process that deposits elongate fibers to form the occlusive membrane 22. The particle size of the fibers creating the occlusive membrane 22 can be configured to permit elongation of the occlusive membrane 22 to at least about 2× (or about 3×, 4×, 5×, or greater) with less force (e.g. between about 25% and about 75% less force) than that of the native material of the same thickness. For example, the particle size of the fibers can be between about 5 microns and about 25 microns. Additional details of the electrospinning process can be found in U.S. Publication No. 2015/0039017, filed Jul. 31, 2014, titled “METHODS AND DEVICES FOR ENDOVASCULAR EMBOLIZATION,” which is included in the Appendix.

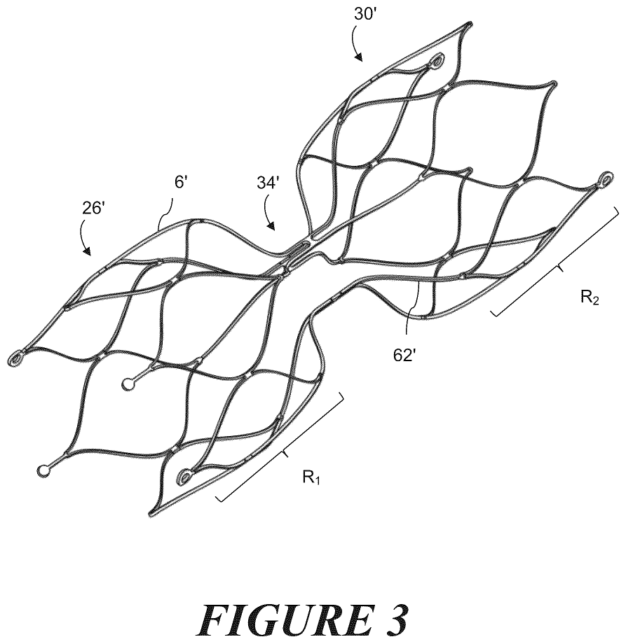

FIG. 3 illustrates an alternative embodiment of a support structure 6′. The support structure 6′ can include any of the features of the support structure 6 described in connection with FIGS. 2A and 2B, except unlike the support structure 6, the struts 62′ connecting the rings R₁′, R₂′ of cells can be interconnected in the neck component 34′ to form additional cells (e.g., diamond-shaped cells) that span the neck component 34′ and either the occlusive component 26′ or the anchoring component 30′.

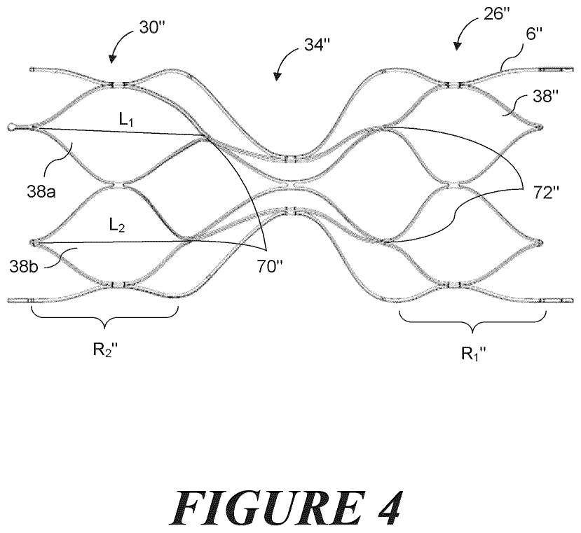

FIG. 4 illustrates yet another embodiment of a support structure 6″. The support structure 6″ can include any of the features of the support structure 6 described in connection with FIGS. 2A and 2B, except as set forth below. Each of the anchoring component 30″ and the occlusive component 26″ can have at least one ring of cells (e.g., one, two, three, or more). Cells 38″ of ring R_{2″} in the anchoring component 30″ can alternate in length of axis, e.g. from an end node to an opposite end node, such that the nodes 70″ closest to the neck component 34″ are both longitudinally and circumferentially offset from each other. Alternating nodes 70″ closest to the neck component can be longitudinally aligned with each other.

As shown in FIG. 4, a length L₁ of a first cell 38a of the anchoring component 30″ is longer than a length L₂ of a circumferentially adjacent second cell 38b of the anchoring component 30″. This pattern can repeat itself around the ring R₂° of the anchoring component 30″ (e.g., for up to four cells, six cells, eight cells, or otherwise as the need may dictate). For example, the ring R_{2″} can include a third cell (not shown) circumferentially adjacent the second cell 38b, on an opposite side of the second cell 38b than the first cell 38a. The third cell can have a same length as the first cell 38a. In contrast, a length of each cell 38″ of ring R_{1″} of the occlusive component 26″ can be generally the same, such that the nodes 72″ on the occlusive component 26″ closest to the neck component 34″ can be circumferentially offset, but longitudinally aligned.

If the nodes 70″ are longitudinally aligned with each other, the stress of retraction is focused at circumferential region of the occlusion device. Thus, staggering the nodes 70″, as shown in FIG. 4, reduces the forces associated with retracting the support structure 6″ into the delivery catheter. The alternating node structure facilitates the retraction of the anchoring component 30″ prior to exposing the occlusive component 26″ to blood flow and beginning the clotting process. Alternatively or additionally, an angle of an apex leading to the nodes 70″ can be varied to distribute the forces associated with retracting the support structure 6″. As another example, strut thickness can be varied to redistribute the forces associated with retraction. For example, struts in the neck component 34″ can be thicker than in the anchoring component 30″.

### Pusher Assembly

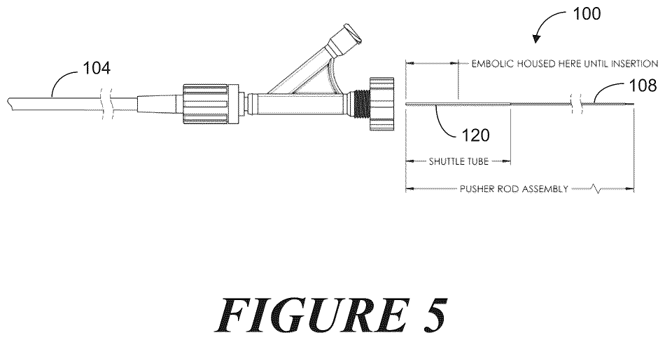

FIG. 5 illustrates a pusher assembly 100 configured to deploy the occlusion device 2 in the neurovasculature under fluoroscopic guidance. The pusher assembly 100 can include an interlock pusher 108 that can deliver the occlusion device 2 through a separate microcatheter 104 or other conduit previously inserted into the patient.

The pusher assembly 100 can have an outer diameter of less than or equal to about: 1.0 mm, 0.75 mm, 0.6 mm, or 0.5 mm, thereby minimizing trauma to the vessel during delivery. The pusher assembly 100 can have a length of at least about 200 cm.

As shown in FIG. 5, the pusher assembly 100 can include a shuttle tube 120. The occlusion device 2 can be pre-positioned in a distal portion of the shuttle tube 120 prior to deployment. The shuttle tube 120 can have an inner diameter of less than or equal to about: 1.0 mm, 0.75 mm, 0.6 mm, or 0.5 mm.

The shuttle tube 120 can be at least partially advanced through the pre-positioned microcatheter 104 or captured at a proximal end of the microcatheter 104. Simultaneously or subsequently, an interlock pusher 108 can advance the occlusion device 2 through the shuttle tube 120 and the microcatheter 104 to deploy the occlusion device 2 at the target vessel.

The pusher assembly 100 can include one or more radiopaque features to facilitate fluoroscopic visualization of the distal end of the pusher assembly 100, and thus a proximal position of the occlusion device 2. For example, a platinum marker band or coiled wire can be positioned at a distal end of pusher assembly 100.

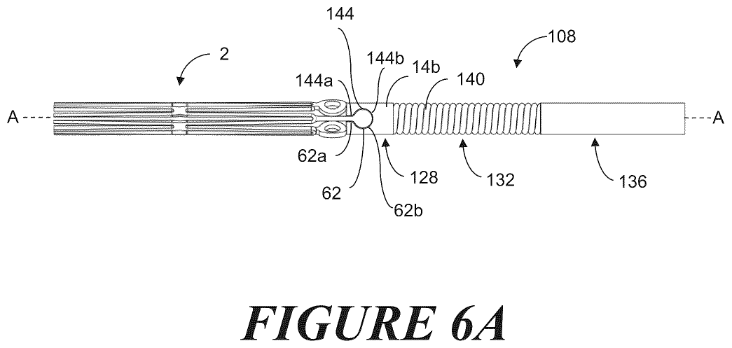

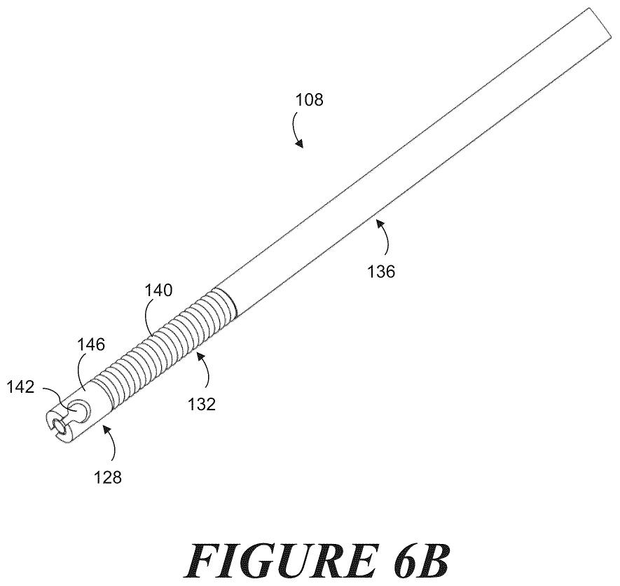

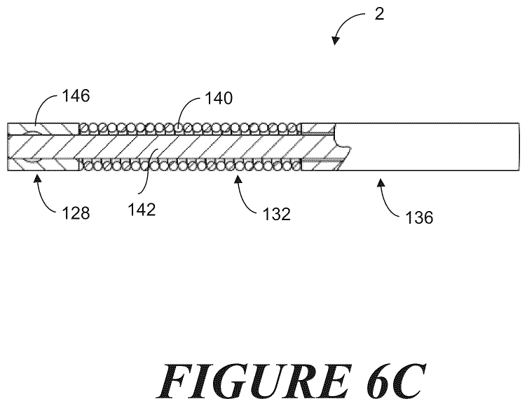

As shown in FIGS. 6A-6C, the interlock pusher 108 can include a distal region 128 configured to interlock with the occlusion device 2. Proximal to the distal region 128, the interlock pusher 124 can include an intermediate region 132, and a proximal region 136. The intermediate region 132 can be more flexible than the proximal region 136 and constructed to facilitate navigation through the neurovasculature. The intermediate region 132 can form less than or equal to about 20% (or less than or equal to about: 18%, 15%, 12%, 10%, or otherwise) of a length of the interlock pusher 124. The intermediate region 132 can include a coil 140 (e.g., a shoulder-shoulder coil). The coil 140 can be formed by a wire (e.g., nitinol wire) having a diameter between about 0.003 inches and about 0.005 inches. An outer diameter of the coil 140 can be less than or equal to about: 0.020 inches, 0.018 inches, 0.015 inches, or otherwise. As shown in FIG. 6B, the coil 140 may be secured over a core wire 142 to prevent the coil from unraveling.

The occlusion device 2 can provide a first interference surface 62 for engaging with a second complementary interference surface 144 of the distal region 128 to releasably retain the occlusion device 2 on the interlock pusher 108 (see FIG. 6A). The first interference surface 62 can be positioned at or displaced from a proximal portion of the occlusion device 2 (e.g., on a proximal features of the support structure 6, on a radiopaque marker 46, or otherwise). The first interference surface 62 can form a unitary structure with the support structure 6, or the interference surface can be attached to the support structure (e.g., by press-fit, welding, adhesives, or otherwise).

As shown in FIG. 6A, the second interference surface 144 can be configured to receive the first interference surface 62. However, in other configurations, the first interference surface 62 can receive the second interference surface 144. The interference surfaces can each include an elongate portion 62a, 144a and an enlarged portion 62b, 144b (e.g., circular portion) having a width that is greater than a width of the elongate portion. The width being measured transverse to axis A of the occlusion device 2 and the interlock pusher 108. For the first interference surface 62, the enlarged portion 62b can be positioned at a proximal end of the occlusion device 2, while for the interlock pusher 108, the enlarged portion 144b can be positioned proximal to the elongate portion 144a. Other interlock features are described in U.S. Publication No. 2015/0039017, filed Jul. 31, 2014, titled “METHODS AND DEVICES FOR ENDOVASCULAR EMBOLIZATION,” which is included in the Appendix.

As shown in FIGS. 6B and 6C, the second interference surface 144 can be formed on a tubular sleeve 146. The sleeve 146 and the coil 140 can each be separately welded, adhered, or otherwise connected to a core wire 142. The sleeve 146 can be positioned over the distal region 128 and distal to the intermediate region 132. Alternatively, the second sleeve 146 can be directly welded, adhered, or otherwise connected to the coil 140.

In an alternative configuration (not shown), the first interference surface 62 can be displaced from a proximal end of the occlusion device 2. The distal region 128 of the interlock pusher 108 can include a slotted disk configured to receive only the elongate portion 62a of the first interference surface 62. The distal region 128 can include a spacer sized to receive the circular portion 62b of the first interference surface. Further, the distal region 128 can further include a proximal stop disk positioned longitudinally between the spacer and the intermediate region 132.

### Method of Use

The occlusion device 2 can be advanced to the target vessel using any of the pusher assemblies described herein. In use, the access to the vasculature can be provided using conventional techniques through an incision on a peripheral artery, such as right femoral artery, left femoral artery, right radial artery, left radial artery, right brachial artery, left brachial artery, right axillary artery, left axillary artery, right subclavian artery, or left subclavian artery. The microcatheter 104 or other introducer structure can be inserted through the access site.

With the occlusion device 2 pre-positioned on the distal region 128 of the interlock pusher 108 and in a distal region of the shuttle tube 120, the pusher assembly 100 can engage and/or be at least partially advanced through the microcatheter 104 until the occlusion device 2 is positioned at a distal end of the microcatheter 104. The shuttle tube 120 and/or the microcatheter 104 can be retracted to expose the occlusion device 2 to blood flow.

The distal anchoring portion 10 can be deployed before the proximal occlusive portion 14 of the occlusion device 2. The bare anchoring portion 10 can at least partially anchor the occlusion device 2 in the vessel before deploying the covered occlusive portion 14, which facilitates precise placement of the occlusion device 2. If the anchoring portion 10 appears improperly positioned (e.g., by examining the position of the markers 46), the occlusion device 2 can be retracted prior to exposing the occlusive portion 14 to blood flow. Further, when the occlusive portion 14 is upstream (i.e., proximal) from the anchoring portion 10, the increase in arterial pressure at the proximal end increases the radially outward forces that can help the occlusion device 2 resist migration.

The occlusion device 2 can be released from the interlock pusher 108 when the first and second interference surfaces 62, 144 are advanced out of the distal end of the microcatheter 104, thus releasing the first interference surface 62 of the occlusion device 2 from the second interference surface 144 of the interlock pusher 108. After the performance assessment, it may be necessary to resheath and reposition the occlusion device 2 to position the occlusion device 2 accurately. The occlusion device 2 can be repositioned so long as the occlusion device 2 has not been released from the interlock pusher 108.

If necessary, the occlusion device 2 can be reinforced using other reinforcing devices or techniques. For example, one or more coils can be deployed within the expandable structure, the expandable structure can be reinforced with an occlusion balloon, and/or the target vessel can be ligated closed.

### Terminology

Although certain embodiments have been described herein with respect to neurovascular occlusion devices, the occlusion devices and pusher assemblies described herein can be deployed elsewhere (e.g., coronary and peripheral vasculature, gastrointestinal tract, ureters, or other lumens).

With respect to the pusher assembly, proximal refers to the direction of the control end of the pusher assembly system and distal refers to the direction of the distal tip.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments.

The terms “comprising,” “including,” “having,” and the like are synonymous and are used inclusively, in an open-ended fashion, and do not exclude additional elements, features, acts, operations, and so forth. In addition, the term “or” is used in its inclusive sense (and not in its exclusive sense) so that when used, for example, to connect a list of elements, the term “or” means one, some, or all of the elements in the list.

The terms “approximately,” “about,” “generally,” and “substantially” as used herein represent an amount close to the stated amount that still performs a desired function or achieves a desired result. For example, the terms “approximately,” “about,” “generally,” and “substantially” may refer to an amount that is within 10% of the stated amount as the context may dictate.

The ranges disclosed herein also encompass any and all overlap, sub-ranges, and combinations thereof. Language such as “up to,” “at least,” “greater than,” “less than,” “between” and the like includes the number recited. Numbers preceded by a term such as “about” or “approximately” include the recited numbers. For example, “about 1 mm” includes “1 mm.”

Any methods disclosed herein need not be performed in the order recited. The methods disclosed herein include certain actions taken by a practitioner; however, they can also include any third-party instruction of those actions, either expressly or by implication.

Although certain embodiments and examples have been described herein, it will be understood by those skilled in the art that many aspects of the occlusion devices and pusher assemblies shown and described in the present disclosure may be differently combined and/or modified to form still further embodiments or acceptable examples. All such modifications and variations are intended to be included herein within the scope of this disclosure. A wide variety of designs and approaches are possible. No feature, structure, or step disclosed herein is essential or indispensable.

Some embodiments have been described in connection with the accompanying drawings. However, it should be understood that the figures are not drawn to scale. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, it will be recognized that any methods described herein may be practiced using any device suitable for performing the recited steps.

For purposes of this disclosure, certain aspects, advantages, and novel features are described herein. It is to be understood that not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Moreover, while illustrative embodiments have been described herein, the scope of any and all embodiments having equivalent elements, modifications, omissions, combinations (e.g., of aspects across various embodiments), adaptations and/or alterations as would be appreciated by those in the art based on the present disclosure. The limitations in the claims are to be interpreted broadly based on the language employed in the claims and not limited to the examples described in the present specification or during the prosecution of the application, which examples are to be construed as non-exclusive. Further, the actions of the disclosed processes and methods may be modified in any manner, including by reordering actions and/or inserting additional actions and/or deleting actions. It is intended, therefore, that the specification and examples be considered as illustrative only, with a true scope and spirit being indicated by the claims and their full scope of equivalents.

### Example Embodiments

The following example embodiments identify some possible permutations of combinations of features disclosed herein, although other permutations of combinations of features are also possible.

1. A neurovascular occlusion device for occluding blood flow in a vessel, comprising:
a support structure, self-expandable from a reduced cross-section for transluminal navigation to an enlarged cross-section for occluding a vessel, the support structure defining a concave occlusive component and an anchoring component separated by a neck component, the anchoring component comprising a ring of cells, the ring of cells comprising a first cell and a circumferentially adjacent second cell, a length of the first cell in a longitudinal direction being greater than a length of the second cell in the longitudinal direction.

2. The neurovascular occlusion device of Embodiment 1, wherein the ring of cells comprises a third cell circumferentially adjacent the second cell, the length of the second cell in the longitudinal direction being shorter than a length of the third cell in the longitudinal direction.

3. The neurovascular occlusion device of Embodiment 1 or 2, wherein the occlusive component comprises a ring of cells, each cell in the at least one ring having a same length.

4. The neurovascular occlusion device of any one of the preceding embodiments, wherein nodes of each of the cells closest to the neck component are longitudinally aligned with each other.

5. The neurovascular occlusion device of any one of the preceding embodiments, wherein the anchoring component is concave in a direction away from the concave occlusive component.

6. The neurovascular occlusion device of any one of the preceding embodiments, wherein the support structure has an expansion ratio of at least about 8:1.

7. The neurovascular occlusion device of any one of the preceding embodiments, wherein the expansion ratio is at least about 10:1.

8. The neurovascular occlusion device of any one of the preceding embodiments, further comprising a functionally occlusive membrane without a guidewire lumen and carried by the occlusive component.

9. The neurovascular occlusion device of Embodiment 8, wherein the functionally occlusive membrane is configured to prevent at least 90% of fluid flow from flowing past the occlusion device at 120 mmHg pressure.

10. The neurovascular occlusion device of any one of the preceding embodiments, further comprising elongate markers positioned at a proximal end and/or a distal end of the occlusion device, a major axis of the elongate markers extending in a longitudinal direction of the occlusion device.

11. The neurovascular occlusion device of Embodiment 10, wherein the elongate markers are rectangular.

12. A neurovascular occlusion device for occluding blood flow in a vessel, comprising:
a support structure, self-expandable from a reduced cross-section for transluminal navigation to an enlarged cross-section for occluding a vessel, the support structure defining a concave occlusive component and an anchoring component separated by a neck component; the anchoring component comprising a ring of cells, each cell comprising a node that is closest to the neck component, the nodes being staggered such that each node is circumferentially and longitudinally offset from the node of a circumferentially adjacent cell and alternating nodes are longitudinally aligned with each other.

13. The neurovascular occlusion device of Embodiment 12, wherein the concave occlusive component comprises a ring of cells, each cell comprising a node closest to the neck component, the nodes being longitudinally aligned with each other.

14. The neurovascular occlusion device of Embodiment 12 or 13, further comprising a functionally occlusive membrane without a guidewire lumen and carried by the occlusive component.

15. The neurovascular occlusion device of Embodiment 14, wherein the functionally occlusive membrane is configured to prevent more than 90% of fluid from flowing past the occlusion device at 120 mmHg pressure within three minutes.

16. The neurovascular occlusion device of Embodiment 14 or 15, wherein the functionally occlusive membrane is porous and has no openings that are greater than a pore size of about 0.2 mm.

17. The neurovascular occlusion device of any one of Embodiments 14 to 16, wherein the functionally occlusive membrane comprises a same thickness along a length of the occlusive membrane.

18. The neurovascular occlusion device of Embodiment 17, wherein the functionally occlusive membrane comprises a region of curvature and a cylindrical region, the cylindrical region having a greater number of layers than the region of curvature.

19. The neurovascular occlusion device of any one of Embodiments 14 to 16, wherein the functionally occlusive membrane comprises a region of curvature and a cylindrical region, a thickness of the region of curvature being greater than a thickness of the cylindrical region.

20. The neurovascular occlusion device of any one of Embodiments 12 to 19, wherein a thickness of the occlusive membrane is no greater than about 30 microns.

21. The neurovascular occlusion device of any one of Embodiments 12 to 20, wherein the anchoring component is concave in a direction away from the concave occlusive component.

22. A method of occluding a vessel, the method comprising:
advancing a pusher assembly through an elongate tubular structure until an occlusion device of any one of Embodiments 1 to 21 is positioned at a distal portion of the elongate tubular structure, the pusher assembly having an outer diameter of less than or equal to about 3F and comprising:
   an interlock pusher configured to be axially advanced through the shuttle tube, the pusher assembly comprising a distal region, an intermediate region, a proximal region, and a lumen extending therethrough;
retracting the elongate tubular structure to release the occlusion device, the occlusion device comprising a first interference surface configured to interface with a second interference surface of the distal region of the interlock pusher until the occlusion device is released from the interlock pusher.

23. The method of Embodiment 22, further comprising retracting the anchoring component of the occlusion device prior to exposing the occlusive portion to blood flow.

24. A system for occluding a vessel, the system comprising:
an elongate tubular structure;
a pusher assembly having an outer diameter of less than or equal to about 3F and comprising:
   an interlock pusher configured to be axially advanced through the shuttle tube, the pusher assembly comprising a distal region, an intermediate region, a proximal region, and a lumen extending therethrough; and
an occlusion device of any one of Embodiments 1 to 21, the occlusion device further comprising a first interference surface configured to interface with a second interference surface of the distal region of the interlock pusher until the occlusion device is released from the interlock pusher.

25. The system of Embodiment 24, wherein the intermediate region of the interlock pusher is more flexible than the proximal region to facilitate navigation through the vessel.

26. The system of Embodiment 25, wherein the intermediate region comprises a coil.

27. The system of any one of Embodiments 24 to 26, wherein the occlusion device further comprises a proximal marker positioned at a proximal end of the support structure, and wherein the proximal marker comprises the first interference surface.

28. The system of any one of Embodiments 24 to 27, wherein the first interference surface is displaced from a proximal end of the occlusion device.

29. The system of any one of Embodiments 24 to 27, wherein the first interference surface is positioned at a proximal end of the occlusion device.

1. A neurovascular occlusion device for occluding blood flow in a vessel, comprising:
a support structure, self-expandable from a reduced cross-section for transluminal navigation to an enlarged cross-section for occluding a vessel, the support structure defining a concave occlusive component and an anchoring component separated by a neck component, the anchoring component comprising a ring of cells, the ring of cells comprising a first cell and a circumferentially adjacent second cell, a length of the first cell in a longitudinal direction being greater than a length of the second cell in the longitudinal direction, the neck component comprising a cell having an open area spanning the neck component; and
a functionally occlusive membrane without a guidewire lumen and carried by the concave occlusive component;
wherein a first ratio of wall space to open area in the neck component is less than a second ratio of wall space to open area in the concave occlusive component or the anchoring component.

2. The neurovascular occlusion device of claim 1, wherein the ring of cells comprises a third cell circumferentially adjacent the second cell, the length of the second cell in the longitudinal direction being shorter than a length of the third cell in the longitudinal direction.

3. The neurovascular occlusion device of claim 1, wherein the occlusive component comprises at least one ring of cells, each cell in the at least one ring having a same length.

4. The neurovascular occlusion device of claim 1, wherein each cell includes nodes, the nodes of each of the cells closest to the neck component are longitudinally aligned with each other.

5. The neurovascular occlusion device of claim 1, wherein the anchoring component is concave in a direction away from the concave occlusive component.

6. The neurovascular occlusion device of claim 1, wherein the support structure has an expansion ratio of at least about 8:1.

7. The neurovascular occlusion device of claim 1, wherein the functionally occlusive membrane is configured to prevent at least 90% of fluid flow from flowing past the occlusion device at 120 mmHg pressure.

8. The neurovascular occlusion device of claim 1, further comprising elongate markers positioned at a proximal end and/or a distal end of the occlusion device, a major axis of the elongate markers extending in a longitudinal direction of the occlusion device.

9. The neurovascular occlusion device of claim 8, wherein the elongate markers are rectangular.

10. A neurovascular occlusion device for occluding blood flow in a vessel, comprising:
a support structure, self-expandable from a reduced cross-section for transluminal navigation to an enlarged cross-section for occluding a vessel, the support structure defining a concave occlusive component and an anchoring component separated by a neck component; the anchoring component comprising a ring of cells, each cell comprising a node that is closest to the neck component, the nodes being staggered such that each node is circumferentially and longitudinally offset from the node of a circumferentially adjacent cell and alternating nodes are longitudinally aligned with each other,
a functionally occlusive membrane without a guidewire lumen and carried by the occlusive component;
wherein the neck component comprises a cell having a first open area, extending from the concave occlusive component to the anchoring component, the first open area being greater than an open area of each cell of the ring of cells.

11. The neurovascular occlusion device of claim 10, wherein the concave occlusive component comprises a ring of cells, each cell comprising a node closest to the neck component, the nodes being longitudinally aligned with each other.

12. The neurovascular occlusion device of claim 10, wherein the functionally occlusive membrane is configured to prevent more than 90% of fluid from flowing past the occlusion device at 120 mmHg pressure within three minutes.

13. The neurovascular occlusion device of claim 12, wherein the functionally occlusive membrane is porous and has no openings that are greater than a pore size of about 0.2 mm.

14. The neurovascular occlusion device of claim 12, wherein the functionally occlusive membrane has a same thickness along a length of the occlusive membrane.

15. The neurovascular occlusion device of claim 10, wherein the functionally occlusive membrane comprises a region of curvature having a first number of layers and a cylindrical region having a second number of layers, the second number of layers of the cylindrical region being greater than the first number of layers of the region of curvature.

16. The neurovascular occlusion device of claim 12, wherein the functionally occlusive membrane comprises a region of curvature and a cylindrical region, a thickness of the region of curvature being greater than a thickness of the cylindrical region.

17. The neurovascular occlusion device of claim 12, wherein a thickness of the functionally occlusive membrane is no greater than about 30 microns.

18. The neurovascular occlusion device of claim 10, wherein the anchoring component is concave in a direction away from the concave occlusive component.

19. The neurovascular occlusion device of claim 10, wherein the first open area is greater than an open area of each cell in the anchoring component.

20. The neurovascular occlusion device of claim 1, wherein the functionally occlusive membrane is applied to an interior of the support structure and an exterior of the support structure.

21. The neurovascular occlusion device of claim 1, wherein the functionally occlusive membrane covers each cell in the concave occlusive component.
